(19) United States
(12) Patent Application Publication
(10) Pub. No.: US 2016/0166594 A1
(43) Pub. Date: Jun. 16, 2016

(54) Dietary Supplement Regimen for Improving Joint Support

(71) Applicant: John BARONE, Morganville, NJ (US)

(72) Inventor: John BARONE, Morganville, NJ (US)

(21) Appl. No.: 14/968,660

(22) Filed: Dec. 14, 2015

Related U.S. Application Data
(60) Provisional application No. 62/091,859, filed on Dec. 15, 2014.

(51) Int. Cl. A61K 31/7008; A61K 31/726

(52) U.S. Cl. CPC A61K 31/7008; A61K 31/726; A23L 1/09; A23L 1/30

(57) ABSTRACT

The instant invention is for a dietary supplement regimen and/or method of improving joint support treatment. As disclosed, the invention concerns administration of an alternating and repeating schedule of administering a primary joint supplement, such as Glucosamine, followed by administering a dose of a secondary joint supplement, such as chondroitin sulfate and vice versa. By dosing supplements and administering incrementally according to the invention, efficacy of the supplements is greatly improved.

This is a non-provisional application claiming priority under 35 USC 119(e) to provisional Application Ser. No. 62/091,859 filed on Dec. 15, 2014, reference of which is incorporated herein in its entirety.

### I. BACKGROUND

A. Field of Invention

The present invention generally relates to dietary supplements. In particular, the present invention is for a dietary supplement and method of delivery of dietary supplements that improves effectiveness in joint support. The invention is a method of delivering a supplement containing two specialized dietary supplements (a Glucosamine Sulfate or Glucosamine Hydrochloride based and a Chondroitin Sulfate) aimed at providing joint support and intended to be taken following the scheduled administration system.

B. Description of the Related Art

Joints are where bones, connective tissue and cartilage all come together in a body to promote movement. The joint support system is responsible for sustaining the area around muscles and bones, where they provide shock absorption and support a body's internal structure.

There are many ways to encourage healthy joint function, like maintaining a healthy weight and enhancing a diet with established joint care supplements and ingredients such as Glucosamine and Chondroitin.

Glucosamine is naturally found within the joints, making it a good supplement for supporting healthy joints and connective tissue. Commonly sold forms of glucosamine are glucosamine sulfate, glucosamine hydrochloride, and N-acetylglucosamine. Glucosamine lubricates the joints to support mobility and flexibility. Glucosamine is naturally present in the shells of shellfish, animal bones, bone marrow, and fungi. In addition, Glucosamine can be sourced from Vegetarian material.

Chondroitin is a naturally occurring nutrient found in connective tissue, and in supplement form, it helps promote healthy cartilage and support the structural framework of joints. It is a chemical that is normally found in cartilage around joints in the body. Chondroitin sulfate is manufactured from animal sources and is typically made from extracts of cartilaginous tissue from cow cartilage, pig, shark, fish, chickens and birds.

Current treatment methods for joint support include administering supplements that contain a blend of Glucosamine and Chondroitin in a single dose, or contain Glucosamine or Chondroitin separately, in a single dose. While the rate of absorption of Chondroitin and Glucosamine by itself may be considered adequate, when the two ingredients are combined, their absorption and overall bioavailability rates drop. Further, while the current treatment methods are considered to be adequate, the efficacy of the supplements can be greatly improved. Additionally, following the instant regimen, the formulations are optimized and the amount of necessary supplements to be taken can be reduced, thus minimizing the financial burden and expense of purchasing supplements and ultimately reducing the long term and/or aggregate unwanted effects associated with the supplements.

### II. SUMMARY

The instant inventor has discovered that the absorption rate and or effectiveness of Glucosamine, the primary joint supplement, is increased when followed by a dose of Chondroitin, the secondary joint supplement, at a predetermined time interval, and vice versa. As a result, the efficacy of the supplements in providing joint support is improved.

Studies show that Chondroitin Sulfate absorbs effectively by itself and Glucosamine absorbs effectively by itself. When the two ingredients are combined however, their absorption rates drop. A purpose of the instant invention is to solve this issue. By separating the ingredients via a scheduled administration system, the absorption rates will remain similar to when the products are taken by themselves.

In addition, the secondary administration will be more effective once some of the primary administration loses effectiveness through the day. By dosing two individual formulations rather than one formulation, such novelty includes separation, never before done in the same joint supplement before. Current supplements are broken into three categories based on the main active ingredient(s): supplements that blend glucosamine and chondroitin into a single dose, glucosamine supplements, and Chondroitin sulfate supplements. By taking these active ingredients (glucosamine and chondroitin) separately (specifically 6 to 18 hours apart) and at two separate time intervals, the results include an improved effectiveness of both active ingredients without the pitfall of absorption rates that blended supplements fall into.

### III. DESCRIPTION OF THE DRAWINGS

The accompanying drawing illustrates the related time tables and efficacy related to the subject invention. In such drawings,

FIG. 1 is a table that provides relative absorption rates of supplements for a combined Glucosamine Hydrochloride and Chondroitin (G.HCl+CS) versus Glucosamine Hydrochloride versus Glucosamine Sulfate (CGS). From this figure, you can see the peaks are formed on all three combinations. In all three combinations, as the day progresses, overall effects begin to wear off. However, by combining (G.HCl+CS), it produces a weaker lasting effect causing it to wear off much faster.

FIG. 2 is the same table from FIG. 1 but has an area highlighted. This identifies, in the shaded region, the effective longevity or peak of a first administration of the corresponding supplements.

FIG. 3 is the same table from FIG. 1 that identifies, in the shaded region, the critical time and or prescribed window of the period of time for administering the secondary dose, as disclosed in the instant invention.

FIG. 4 is a daily illustration of how a single administration would be. From the illustration, it is clear that toward the middle and end of the day, overall effectiveness begins to wear off. This is what is typically found in the market today.

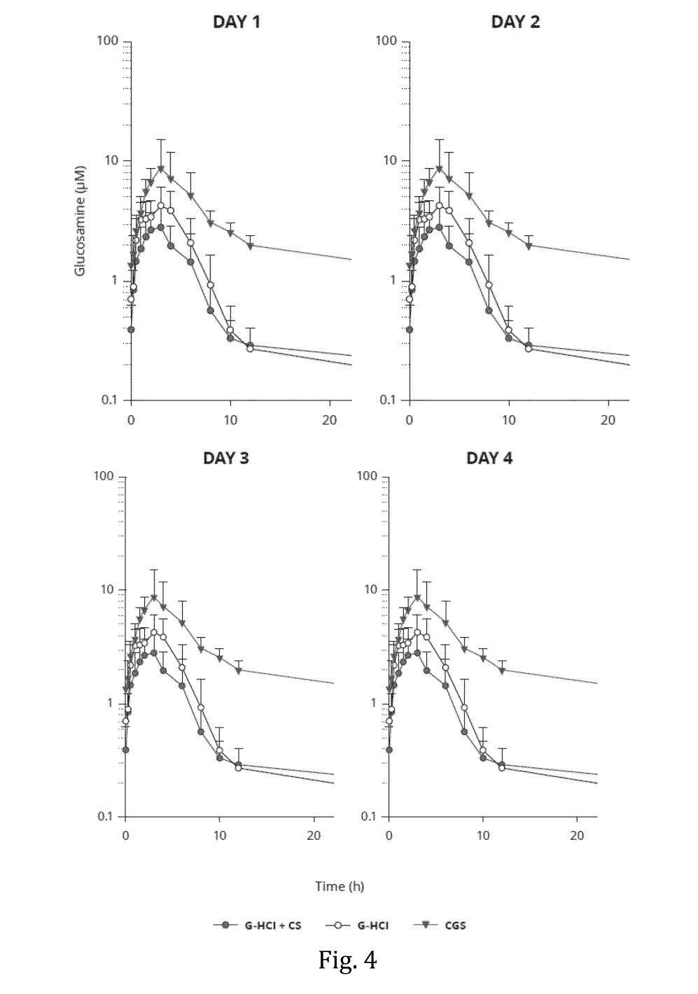

FIG. 5 is a modified graph simply doubling the data points from FIG. 1 to loosely illustrate how the instant invention would work on a daily basis. The invention is intended to give two peaks during the same 24-hour period, utilizing the same quantity, introducing the secondary dose before primary dose wears off.

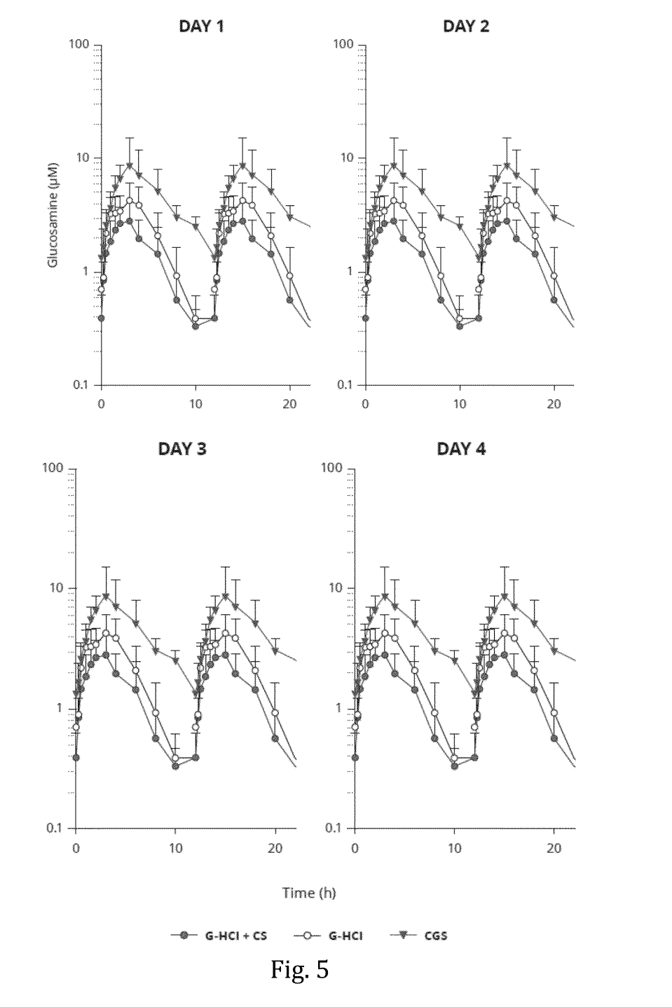

FIG. 6 are tables highlighting the peak for chondroitin sulfate dose.

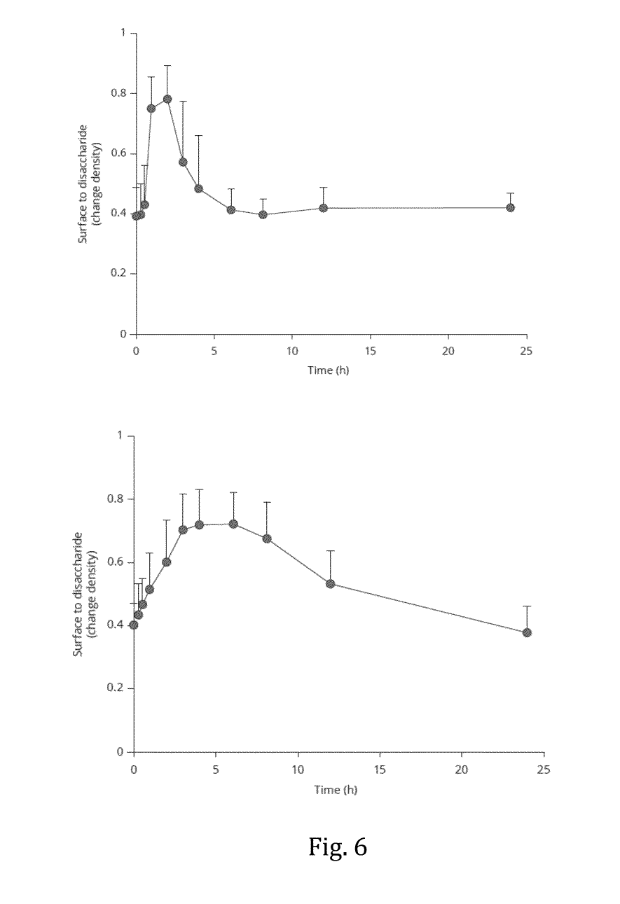

FIG. 7 is a table with data points highlighting the effects of combining Glucosamine Hydrochloride and Chondroitin (G.HCl+CS) and how it compares to the Glucosamine Hydrochloride and the Glucosamine Sulfate (CGS).

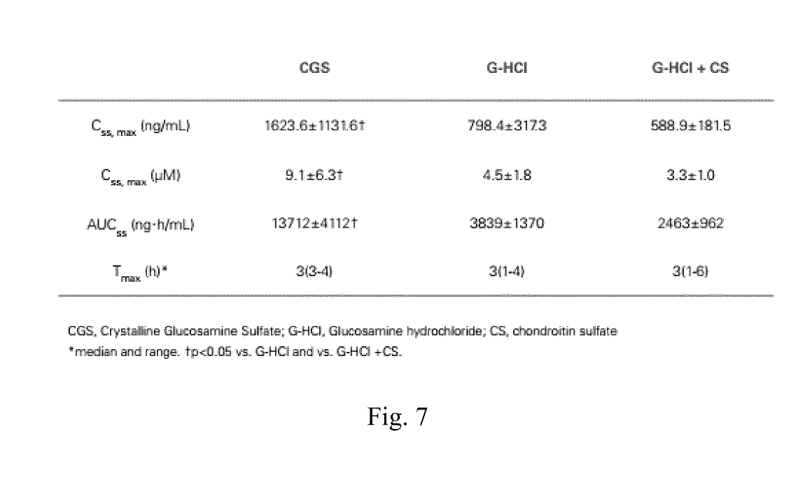

FIG. 8 is a list of possible additions (individually or in combination) to either formulation, whether it be in the Primary or Secondary Formulation.

### IV. DETAILED DESCRIPTION OF THE INVENTION

As shown in the accompanying drawings, for purposes of illustration, and explained herein, the present invention resides in dietary supplements (primary and secondary formulations or dosages) and methods of delivery thereof for providing joint support.

In an embodiment, the primary joint supplement can be either Glucosamine, Glucosamine Hydrochloride, Glucosamine Sulfate and/or N-Acetylglucosamine and the secondary joint supplement, administered in accordance with the instant invention can be Chondroitin Sulfate and the lecticans, components of the extracellular matrix. In another embodiment, the order of the primary and secondary supplements as indicated above may be reversed.

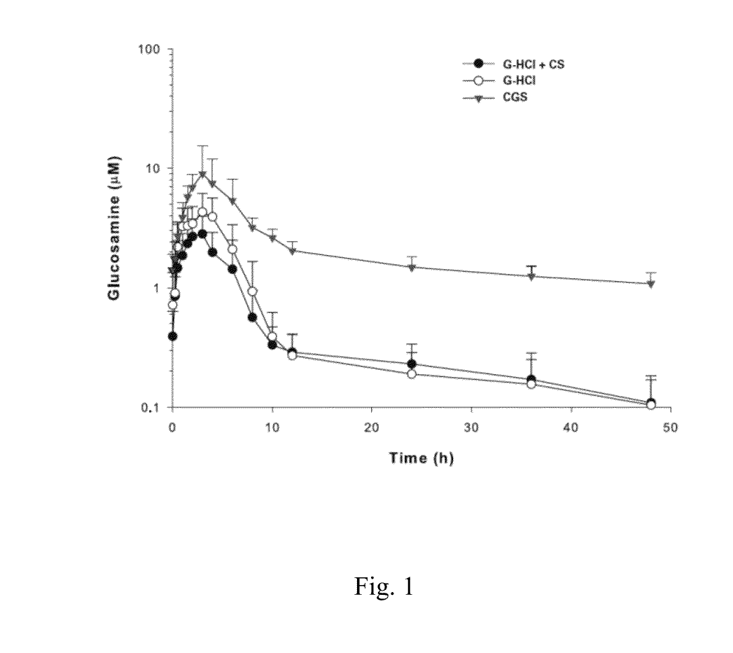

FIG. 1 is a table that provides relative absorption rates of combined Glucosamine Hydrochloride and Chondroitin (G.HCl+CS) versus Glucosamine Hydrochloride versus Glucosamine Sulfate (CGS) after repeated doses. As shown, the efficacy of Glucosamine Sulfate is comparatively superior to Glucosamine Hydrochloride and Chondroitin (G.HCl+CS) versus Glucosamine Hydrochloride. Notwithstanding, over the course of a 24 hour period, the absorption of glucosamine experiences a failure and or drastic declination. As a direct consequence of the failure to maintain absorption, the effectiveness of the supplement in providing joint support likewise declines and fails to provide customers with extended benefits

It has been discovered that the rate of absorption of Glucosamine will be maximized over time, defined by cycles, when a first cycle is defined by an initial dose of a primary joint supplement at time, t₀, followed by a dose of a secondary joint supplement that is administered during a critical time range after, t_{0c}. Then, following completion of a first cycle, a subsequent dose of a primary joint supplement is introduced thereafter, defining a second cycle, which is administered at a critical time range, t_{1c}, followed by a dose of a secondary joint supplement that is administered during a critical time range, t_{2c}. The cycles repeat, whereby subsequent, alternating doses (primary-to-secondary) are administered at critical times, until optimum results are realized.

It should be understood that the instant invention includes the embodiment where the first cycle may also be defined by an initial dose of a secondary joint supplement at time, t₀, followed by a dose of a primary joint supplement that is administered during a critical time range after, t_{0c}. Then, following completion of a first cycle, a subsequent dose of a secondary joint supplement is introduced thereafter, defining a second cycle, which is administered at a critical time range, t_{1c}, followed by a dose of a primary joint supplement that is administered during a critical time range, t_{2c}.

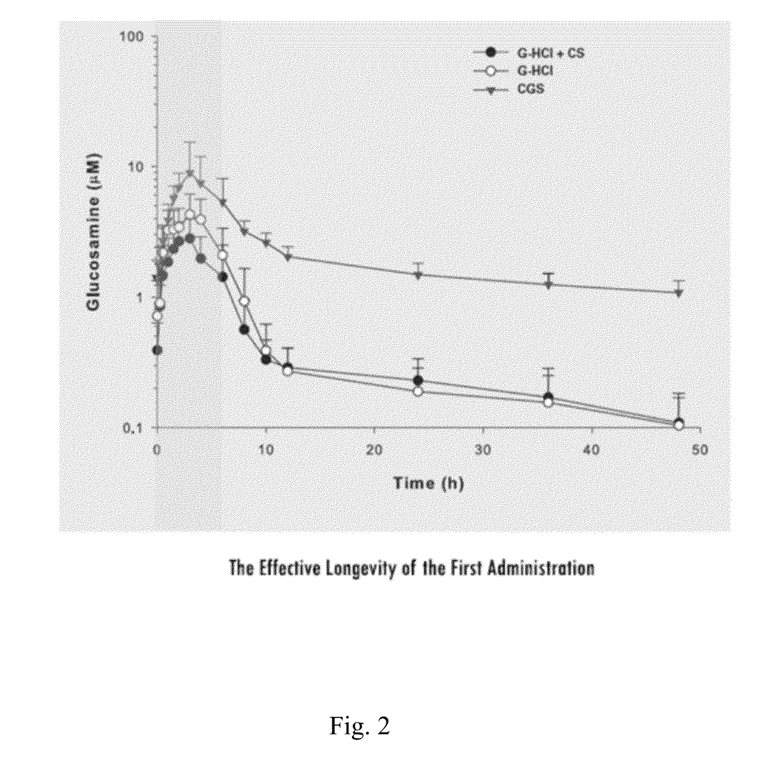

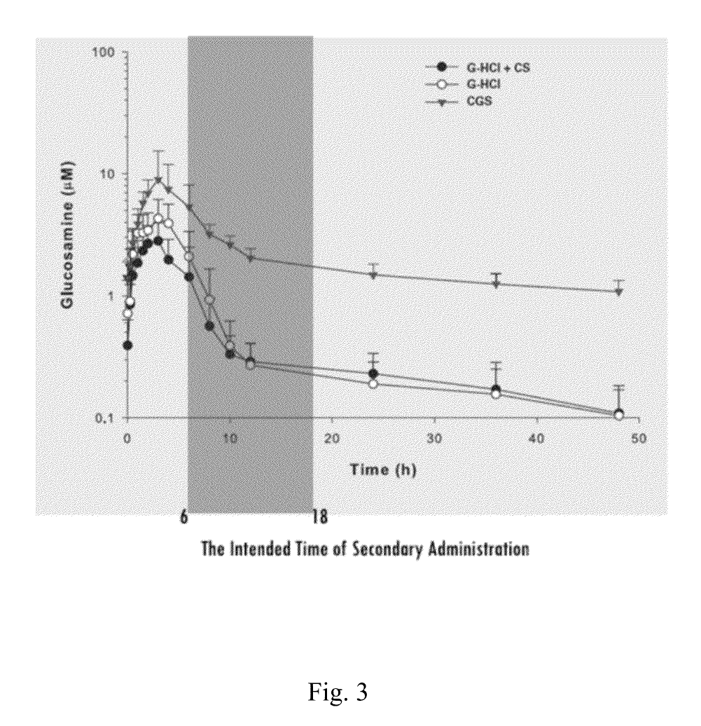

As shown in FIG. 2, the shaded region defines efficacy and or effective longevity of a first administration of corresponding supplements. Depending on the longevity of the supplement administered in the first cycle, the first critical time, t_{0c}, may have de minimus and or slight variance. FIG. 3 identifies, in the shaded region, the prescribed critical window of the period of time for administering the secondary dose, i.e., at the first critical time, t_{0c}.

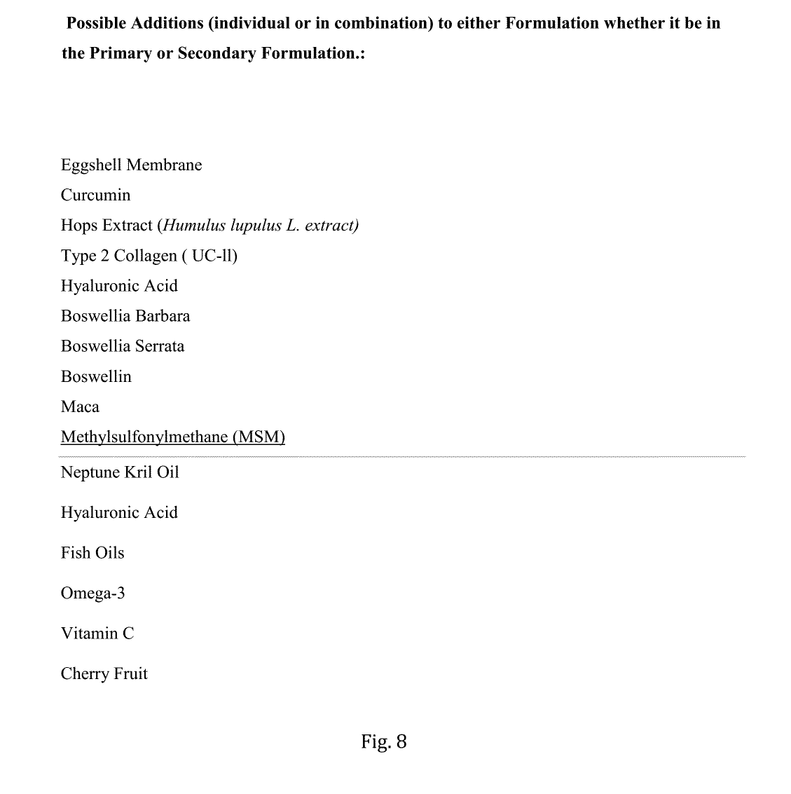

As provided for herein, an example of the suggested administration of supplements according to the instant invention include,
t₀: administering dose of primary joint supplement, whether it be Glucosamine, Glucosamine Hydrochloride, Glucosamine Sulfate, and/or N-Acetylglucosamine. In addition, any ingredients from FIG. 8 (whether individually or in combination) can be added to the primary joint supplement.
t_{0c}: administering dose of secondary joint supplement, chondroitin sulfate. In addition, any ingredients from FIG. 8 (whether individually or in combination) can be added to the primary joint supplement.
t_{1c}: administering dose of primary joint supplement, whether it be Glucosamine, Glucosamine Hydrochloride, Glucosamine Sulfate, and or N-Acetylglucosamine. In addition, any ingredients from FIG. 8 (whether individually or in combination) can be added to the primary joint supplement.
t_{2c}: administering dose of secondary joint supplement, chondroitin sulfate. In addition, any ingredients from FIG. 8 (whether individually or in combination) can be added to the primary joint supplement.
t_{3c}: administering dose of primary joint supplement, whether it be Glucosamine, Glucosamine Hydrochloride, Glucosamine Sulfate, and or N-Acetylglucosamine. In addition, any ingredients from FIG. 8 (whether individually or in combination) can be added to the primary joint supplement.
t_{ic}: administering dose of primary joint supplement, whether it be Glucosamine, Glucosamine Hydrochloride, Glucosamine Sulfate, and or N-Acetylglucosamine. In addition, any ingredients from FIG. 8 (whether individually or in combination) can be added to the primary joint supplement. when i is an odd number)
t_{i+1c}: administering dose of secondary joint supplement, chondroitin sulfate. In addition, any ingredients from FIG. 8 (whether individually or in combination) can be added to the primary joint supplement. (when i is an odd number)

The above example of the suggested administration of supplements according to the instant invention can be reversed to include,
t₀: administering dose of primary joint supplement, chondroitin sulfate. In addition, any ingredients from FIG. 8 (whether individually or in combination) can be added to the primary joint supplement.
t_{0c}: administering dose of secondary joint supplement, whether it be Glucosamine, Glucosamine Hydrochloride, Glucosamine Sulfate, and or N-Acetylglucosamine. In addition, any ingredients from FIG. 8 (whether individually or in combination) can be added to the primary joint supplement.
t_{1c}: administering dose of primary joint supplement, chondroitin sulfate. In addition, any ingredients from FIG. 8 (whether individually or in combination) can be added to the primary joint supplement.
t_{2c}: administering dose of secondary joint supplement, whether it be Glucosamine, Glucosamine Hydrochloride, Glucosamine Sulfate, and or N-Acetylglucosamine. In addition, any ingredients from FIG. 8 (whether individually or in combination) can be added to the primary joint supplement.
t_{3c}: administering dose of primary joint supplement, chondroitin sulfate. In addition, any ingredients from FIG. 8 (whether individually or in combination) can be added to the primary joint supplement.
t_{ic}: administering dose of primary joint supplement, chondroitin sulfate. In addition, any ingredients from FIG. 8 (whether individually or in combination) can be added to the primary joint supplement. when i is an odd number)

t_{i+1c}: administering dose of secondary joint supplement, whether it be Glucosamine, Glucosamine Hydrochloride, Glucosamine Sulfate, and or N-Acetylglucosamine. In addition, any ingredients from FIG. 8 (whether individually or in combination) can be added to the primary joint supplement. (when i is an odd number)

The inventor of the instant invention has discovered that the amount of Glucosamine to be administered per dose may be between the range of about 500 mg to about 2000 mg, and that the subsequent critical time to administer the secondary joint supplement may generally be in the range of about 6 hours to about 18 hours after dose.

The inventor of the instant invention has discovered that the amount of chondroitin sulfate to be administered per dose may be between the range of about 400 mg to about 1200 mg, and that the subsequent critical time to administer the primary joint supplement may generally be in the range of about 6 hours to about 18 hours after dose.

By attending to the regimen as disclosed herein, efficacy of Glucosamine and Chondroitin in joint treatment will be appreciatively improved. Additionally, by alternating primary and secondary supplements, it is understood that the instant invention maximize the effectively of the supplements and that with time less supplements will ultimately be required, thus reducing long term and/or aggregate effects.

Having thus described the invention, what is claimed as new and desired to be secured by Letters Patent is:

1. A dietary supplement regimen for improving joint support, comprising the steps of, (i) administering a dose of a primary joint supplement, then (ii) administering a dose of a secondary joint supplement at a critical time, then (iii) administering a dose of a primary joint supplement at a critical time, and (iv) repeating steps (ii) and (iii) until desired results are achieved.

2. The dietary supplement regimen for improving joint support as provided for in claim 1, whereby the primary joint supplement is a single supplement selected from the group consisting of Glucosamine, Glucosamine Hydrochloride, Glucosamine Sulfate and N-Acetylglucosamine.

3. The dietary supplement regimen for improving joint support as provided for in claim 2, whereby the amount of the primary supplement provided for in a dose is between the range of about 500 mg to about 2000 mg.

4. The dietary supplement regimen for improving joint support as provided for in claim 1, whereby the primary joint supplement is a single supplement selected from the group consisting of Chondroitin Sulfate and lecticans.

5. The dietary supplement regimen for improving joint support as provided for in claim 4, whereby the amount of the primary supplement provided for in a dose is between the range of about 400 mg to about 1200 mg.

6. The dietary supplement regimen for improving joint support as provided for in claim 2, whereby the secondary joint supplement is a single supplement that is selected from the group consisting of Chondroitin Sulfate and lecticans.

7. The dietary supplement regimen for improving joint support as provided for in claim 6, whereby the amount of the secondary joint supplement provided for in a dose is between the range of about 400 mg to about 1200 mg.

8. The dietary supplement regimen for improving joint support as provided for in claim 4, whereby the secondary joint supplement can also be a single supplement selected from the group consisting of Glucosamine, Glucosamine Hydrochloride, Glucosamine Sulfate and N-Acetylglucosamine.

9. The dietary supplement regimen for improving joint support as provided for in claim 8, whereby the amount of the secondary joint supplement provided for in a dose is between the range of about 500 mg to about 2000 mg.

10. The dietary supplement regimen for improving joint support as provided for in claim 1, whereby the critical time for administering a dose of a secondary joint supplement is between about 6 hours to about 18 hours after dosing of the prior, primary joint supplement.

11. The dietary supplement regimen for improving joint support as provided for in claim 1, whereby the critical time for administering a subsequent dose of a primary joint supplement is between about 6 hours to about 18 hours after dosing of the prior, second joint supplement.
